(19) United States
(12) Patent Application Publication
(10) Pub. No.: US 2007/0106341 A1
(43) Pub. Date: May 10, 2007

(54) Therapeutic animal collar

(76) Inventors: Rufus C. Moss, Lillie, LA (US); Elaine Moss, Lillie, LA (US)

Correspondence Address: Galasso & Associates, LP, P.O. Box 26503, Austin, TX, 78755-0503, US

(21) Appl. No.: 11/591,813

(22) Filed: Nov. 2, 2006

Related U.S. Application Data
(60) Provisional application No. 60/732,876, filed on Nov. 2, 2005.

(51) Int. Cl. A61N 1/34

(52) U.S. Cl. 607/46

(57) ABSTRACT

A therapeutic collar designed to faciliate the alleviation of pain in household animals such as a dogs and cats. The therapeutic collar includes an elongated and flexible body, having two ends. The two ends are configured to be releasably serucred to each other in order to be releasably secured the collar about the neck area of a household animal. A plurality of electrodes are integrated into the body and are operable to produce at least one of three modes of electrotherapy. The electrodes can be operated to produce microcurrent therapy, transcutaneous electric nerve stimulation and inferential current therapy in order to provide pain management technique for the household animal.

### PRIORITY UNDER 35 U.S.C. 119(e) & C.F.R. 1.78

This nonprovisional application claims priority based upon the following prior United States provisional patent application entitled: Therapy Collar for Pets, Application No.: 60/732,876, filed Nov. 2, 2005, in the names of Rufus C. Moss and Elaine Moss, which is hereby incorporated by reference for all purposes.

### FIELD OF THE INVENTION

The present invention relates to a pain management device for animals, more specifically but not by way of limitation, a collar that is designed to be proximate the neck of an animal such as a dog or cat and is configured to utilize a plurality of electrotherapeutic technologies to alleviate pain in the animal.

### BACKGROUND

Millions of individuals have an animal as a household pet. The most common are dogs and cats. Pet owners are responsible for the continued care and feeding of their pets. Routine care can consist of feeding, bathing and exercising the pet properly. Healthcare is also a responsibility of the pet owner. Pet owners spend billions of dollars in veterinary care each year treating a plurality of illness and maladies of their pets. Whether it is providing post-operative care or treating an injury, a common task of healthcare of a pet is pain management.

One problem with pain management is that the most routine method of treating pain involves narcotics. As pain symptoms can endure for many months or years in some animals, this method is undesirable by some pet owners as the narcotics can have side effects and be expensive to administer for any period of time.

Accordingly, there is a need for a device that can assist a pet owner with the pain management of an animal that can be used long term without negative side effects or extensive costs of the current methods utilizing narcotics.

### SUMMARY OF THE INVENTION

It is the object of the present invention to provide a device for administering pain management for animals that utilizes at least one of a plurality of types of electrotherapy.

A further object of the present invention is to provide a device for administering pain management for animals that uses inferential current as a treatment to alleviate pain.

Yet another object of the present invention is to provide a device for administering pain management for animals that uses microcurrent therapy to alleviate pain.

A further object of the present invention is to provide a device for administering pain management for animals that uses transcutaneous electrical nerve stimulation to alleviate pain.

Yet another object of the present invention is to provide a device for administering and delivering the electrotherapeutic pain management through integrating the electrotherapy such that it can be delivered with a dog collar.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawing wherein:

FIG. 1 is a perspective view of an embodiment of the present invention place on the neck of a dog;

FIG. 2 is a top view of an embodiment of the present invention; and

FIG. 3 is a perspective view of an alternative embodiment of the present invention.

### DETAILED DESCRIPTION

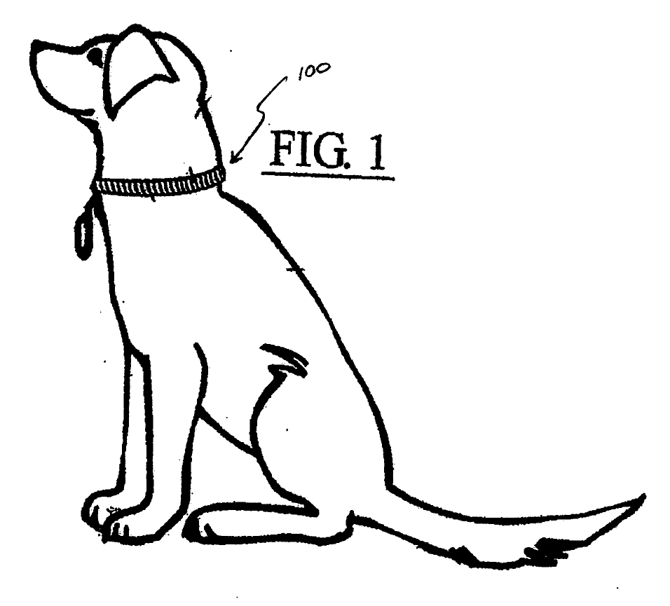

Referring now to the drawings submitted herewith wherein the various elements depicted therein are not necessarily drawn to scale and wherein like elements are identified with like reference numerals and in particular to FIGS. 1 through 3, there is illustrated a preferred embodiment of a therapeutic collar 100 constructed according to the principles of the present invention.

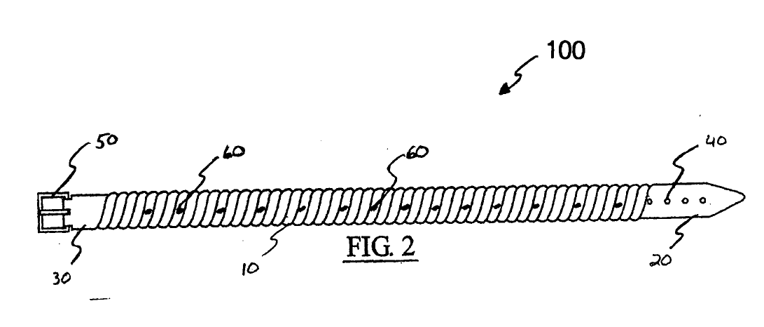

Referring in particular to FIGS. 1 and 2, the therapeutic collar 100 comprises a strap or body 10 that is generally elongated and rectangular in shape. The body 10 is manufactured from a durable and flexible material such as but not limited to leather or plastic. Those skilled in the art will recognize that numerous different materials could be used to manufacture the body 10. Proximate a first end 30 of the body 10 is a buckle 50. The buckle 50 is manufactured to releasably couple with the apertures 40 that are proximate the second end 20 of the body 10 subsequent to the therapeutic collar 100 being secured around and proximate the neck area of the animal. It is contemplated within the scope of the present invention that the therapeutic collar 100 could be utilized on dogs or cats in providing an option for pain management. Those skilled in the art will recognize that the therapeutic collar 100 could be utilized on numerous different animals in order to provide an option for treating pain.

Disposed along the body 10 are a plurality of electrodes 60 that function to provide electrical stimulation to the area generally adjacent thereto. The electrical current generated by the electrodes 60 function to provide pain relief and improved muscle performance. The electrodes 60 are conventional electrodes as is known in the art and are secured to the therapeutic collar by suitable mechanical or chemical methods. Those skilled in the art will recognize that any number of electrodes 60 could be secured to the body 10. Although not shown in the drawing submitted herewith, the electrodes 60 are operably coupled to a power supply such as but not limited to a battery.

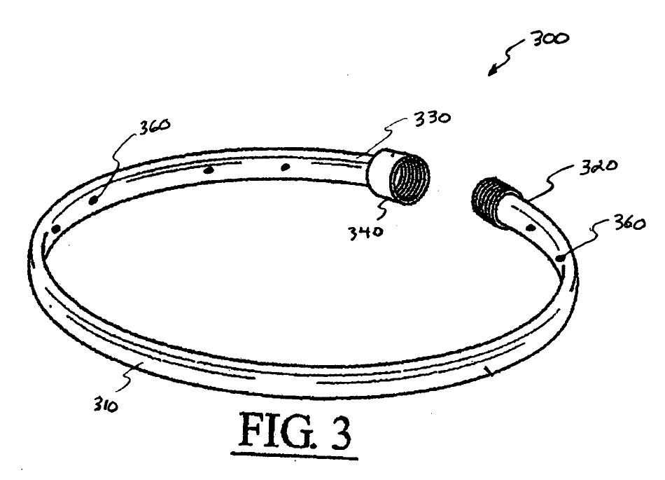

Referring in particular to FIG. 3, an alternative embodiment of the present inventions is illustrated as therapeutic collar 300. The therapeutic collar 300 comprises a body 310 that is generally smooth in texture being elongated and generally arcuate in shape. The body 310 is manufactured from a suitable durable and flexible material so that it can be spread apart to be placed generally around the neck area of an animal. The body 310 has a first end 320 with a threaded portion and a second end 330. Second end 330 includes a fastener 340 configured to receive the threaded portion of second end 330. The fastener 340 functions to releasably couple the first end 320 and the second end 330 once the therapeutic collar 300 has been placed adjacent to the neck area of an animal. Those skilled in the art will recognize that numerous different types of fasteners 340 could be mounted to the body 310.

Disposed along the body 310 are a plurality of electrodes 360. The electrodes 360 provide relief from pain in the adjacent area thereto by a generated electrical current. It has been shown that applying suitable amounts of electrical current reduces swelling and inflammation of the muscle area and speeds the healing process while improving soft tissue regeneration.

Although not limiting, good results have been achieved by utilizing at least one of three different types of electrotherapy. A first type of electrotherapy is micorcurrent therapy. Microcurrent therapy produces electrical signals similar to those naturally occurring when the body is repairing damaged tissues. The electrical signals are applied to the animal with the electrodes located on the collar. By generating electrical currents similar to those naturally occurring in the body, the healing process of the animal is enhanced. While no particular symptom or ailment is required to utilize microcurrent therapy, good results have been achieved when utilizing the therapeutic collar to deliver microcurrent therapy when the animal has soft tissue inflammation or muscle spasms.

A second method of electrotherapy that the therapeutic collar is configured to deliver via the electrodes is transcutaneous electric nerve stimulation. During transcutaneous electric nerve stimulation the electrodes apply small electrical stimuli in order to occupy nerve pathways that transport pain messages. This treatment technique interferes with the transmission of pain messages to the brain and is helpful with treating animals that have chronic pain symptoms.

A third method of electrotherapy that the therapeutic collar is configured to deliver via the electrodes is inferential current therapy. In this method, the electrodes produce a low frequency current at approximately 4000 Hz, which reduces the resistance of the skin. The low frequency current has been shown to increase muscle stimulation in innervated muscles and provide a short term analgesic effect. This treatment mode has further been shown to increase tissue and vessel tonus plus lymphatic and venous blood flow. Those skilled in the art will recognize that the therapeutic collar could be configured to utilize as few as one of the methods of electrotherapy referenced herein or any combination thereof.

It is contemplated within the scope of the present invention, that the therapeutic collars illustrated herein, could further include a power supply and a switch to allow a user to select between the modes of electrotherapy to apply to the desired animal.

Referring in particular to FIG. 2, a description of the operation of the therapeutic collar 100 is as follows. In use, a user will releasably couple the first end 20 to the second end 30 of the body 10 with the buckle 50 generally around and adjacent to the neck area of the animal that has been diagnosed with muscle pain or similar symptom. When engaged with the animal, the electrodes 60 will be activated to generate a method of electrotherapy as referenced herein. The electrotherapy will treat the symptoms of the animal for the desired length of time. The user will then remove the therapeutic collar 100 from the animal's neck area and store for subsequent re-use.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A therapeutic device for an animal, comprising:
a body, said body being generally elongated in shape, said body further being flexible; and
a plurality of electrodes integrated with said body, said plurality of electrodes operable to provide stimulus to the animal.

2. The therapeutic device as recited in claim 1, wherein said plurality of electrodes are operable in at least one of a plurality of electrotherapy modes.

3. The therapeutic device as recited in claim 2, wherein one of said plurality of electrotherapy modes is a microcurrent therapy mode.

4. The therapeutic device as recited in claim 2, wherein one of said plurality of electrotherapy modes is a transcutaneous electrical nerve stimulation mode.

5. The therapeutic device as recited in claim 2, wherein one of said plurality of electrotherapy modes is an inferential current therapy mode.

6. The therapeutic device as recited in claim 2, and further including a fastener such that said body is operable to be releasably secured about the neck area of the animal.

7. A therapeutic device configured to facilitate pain management for an animal, comprising:
a body, said body having a first end and a second end, said first end and said second end being configured to be releasably coupled, said body being rectangular and elongated in shape; and
at least two electrodes, said electrodes being integrated with said body, said electrodes being operable to produce stimuli.

8. The therapeutic device as recited in claim 7, wherein said at least two electrodes are operable in at least one of a plurality of electrotherapy modes.

9. The therapeutic device as recited in claim 8, wherein one of said plurality of electrotherapy modes is a microcurrent therapy mode.

10. The therapeutic device as recited in claim 9, wherein a second of said plurality of electrotherapy modes is a transcutaneous electrical nerve stimulation mode.

11. The therapeutic device as recited in claim 10, wherein a third of said plurality of electrotherapy modes is a transcutaneous electrical nerve stimulation mode.

12. The therapeutic device as recited in claim 8, and further including a fastener to facilitate said first end being releasably coupled to said second end.

13. A therapeutic collar configured to facilitate pain management for household animals comprising:
a body, said body having a first end and a second end, said first end and said second end being configured to be releasably coupled, said body being rectangular and elongated in shape; and
a plurality of electrodes, said plurality of electrodes integrated with said body, said plurality of electrodes operable to provide stimulus to the animal.

14. The therapeutic collar as recited in claim 13, wherein said plurality of electrodes are operable in at least one of a plurality of electrotherapy modes.

15. The therapeutic collar as recited in claim 13, wherein one of said plurality of electrotherapy modes is a microcurrent therapy mode.

16. The therapeutic collar as recited in claim 14, wherein one of said plurality of electrotherapy modes is a microcurrent therapy mode.

17. The therapeutic collar as recited in claim 16, wherein a second of said plurality of electrotherapy modes is a transcutaneous electrical nerve stimulation mode.

18. The therapeutic collar as recited in claim 17, wherein a third of said plurality of electrotherapy modes is an inferential current therapy mode.

19. The therapeutic collar as recited in claim 18, wherein said therapeutic collar is configured to releasably secure to the neck of the household animal.

20. The therapeutic collar as recited in claim 19, wherein the body is operable to interface with a power supply to supply current to said plurality of electrodes.
